(19) United States
(12) Patent Application Publication
(10) Pub. No.: US 2005/0227239 A1
(43) Pub. Date: Oct. 13, 2005

(54) Microarray based affinity purification and analysis device coupled with solid state nanopore electrodes

(76) Inventor: Timothy H. Joyce, Mountain View, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/821,239

(22) Filed: Apr. 8, 2004

(51) Int. Cl. C12Q 1/68; C12M 1/34

(52) U.S. Cl. 435/6; 435/287.2

(57) ABSTRACT

An apparatus and method for separating and identifying chemical moieties. The apparatus employs a microarray device coupled to a nanopore system. The apparatus both separates and identifies target molecules without the requirement of extraneous tags or fluorescent markers. Methods for using the apparatus are also disclosed.

### FIELD OF THE INVENTION

The invention relates to the field of microarrays and more particularly to an apparatus and method for separating, identifying and quantitating chemical moieties using arrays.

### BACKGROUND OF THE INVENTION

Polynucleotide arrays (such as DNA or RNA arrays) are known and are used, for example, as diagnostic or screening tools. Such arrays include regions of usually different sequence polynucleotides arranged in a predetermined configuration on a substrate. These regions (sometimes referenced as “features”) are positioned at respective locations (“addresses”) on the substrate. In use, the arrays, when exposed to a sample, will exhibit an observed binding or hybridization pattern. This binding pattern can be detected upon interrogating the array. For example, all polynucleotide targets (for example, DNA) in the sample can be labeled with a suitable label (such as a fluorescent dye), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Biopolymer arrays can be fabricated by depositing previously obtained biopolymers (such as from synthesis or natural sources) onto a substrate, or by in situ synthesis methods. Methods of depositing obtained biopolymers include dispensing droplets to a substrate from dispensers such as pin or capillaries (such as described in U.S. Pat. No. 5,807,522) or such as pulse-jets (such as a piezoelectric inkjet head, as described in PCT publications WO 95/25116 and WO 98/41531, and elsewhere). For in situ fabrication methods, multiple different reagent droplets are deposited from drop dispensers at a given target location in order to form the final feature (hence a probe of the feature is synthesized on the array substrate). The in situ fabrication methods include those described in U.S. Pat. No. 5,449,754 for synthesizing peptide arrays, and described in WO 98/41531 and the references cited therein for polynucleotides. The in situ method for fabricating a polynucleotide array typically follows, at each of the multiple different addresses at which features are to be formed, the same conventional iterative sequence used in forming polynucleotides from nucleoside reagents on a support by methods of known chemistry. This iterative sequence is as follows: (a) coupling a selected nucleoside through a phosphite linkage to a functionalized support in the first iteration, or a nucleoside bound to the substrate (i.e. the nucleoside-modified substrate) in subsequent iterations; (b) optionally, but preferably, blocking unreacted hydroxyl groups on the substrate bound nucleoside; (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in a known manner.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, and elsewhere.

As discussed above, there are a number of techniques for constructing microarrays. In addition, microarrays may be used to identify and quantitate different types of RNA, DNA or protein molecules in a sample. A microarray comprises a number of surface bound molecules that may be arranged in defined locations. For instance, a sample containing an unknown target is often labeled with a fluorescent dye, applied to the array and allowed to react or hybridize to a probe over a period of time. The array is then washed to remove unbound or inappropriately bound sample and scanned for fluorescent signal. The detected signal at each location is correlated to the probe identity.

In the above example, the array provides a few major functions. The first function is that it acts as a separation device that organizes molecules from the sample into known locations and allows the remainder to be discarded. Second, it is a platform to analyze how many sample molecules were detected at each location. The two functions are independent and each confers its own requirements on the assay design.

The separation function requires that the known probe molecule be attached to the surface in a known or defined location. The pattern of features can be in the form of a grid or a linear arrangement. The detection of these hybridizations is due largely to the use of fluorescent dyes coupled to target molecules. Labeling is typically performed during a sample preparation process that can add significant time to the assay completion. Secondly, the use of labels increases costs, and can potentially cross react with other molecules or probes. Therefore, there is a need for an array system, apparatus or technique that eliminates the need for using labels. There is also a need for such apparatus or method to provide a high level of specificity and reproducibility for identifying and separating small sample volumes or quantities.

Microarrays also suffer from the limitation that they can require multiple runs and may require extensive time to employ in an analysis. In addition, they may be limited by hybridization parameters such as requiring a 20 mer or smaller to obtain complete hybridizations. Each of these requirements, therefore, influences the effectiveness of microarrays as clinical or diagnostic devices. Therefore, there is a need for analytical devices to be able to separate and identify targets at high speeds.

In contrast, nanopore technologies are now being developed to sequence genomes and nucleic acids, or proteins at high speeds. These techniques attempt to sequence the nucleic acid or protein when it passes through a defined nanopore or structure. Manipulating matter at the nanometre (nm) scale is important for many electronic, chemical and biological advances (See Li et al., “Ion beam sculpting at nanometer length scales”, Nature, 412: 166-169, 2001).

It has been demonstrated that a voltage gradient can drive single-stranded biopolymers through a transmembrane channel, or nanopore (See Kasianowicz et al., “Characterization of individual polynucleotide molecules using a membrane channel”, Proc. Natl. Acad. Sci. USA, 93: 13770-13773, 1996). During the translocation process, the extended biopolymer molecule will block a substantial portion of the otherwise open nanopore channel. This blockage leads to a decrease in the ionic current flow of the buffer solution through the nanopore during the biopolymer translocation. The passage of a single biopolymer can be monitored by recording the translocation duration and the blockage current, yielding plots with predictable stochastic sensing patterns. From the uniformly controlled translocation conditions, the lengths of the individual biopolymers can be determined from the translocation time. Furthermore, the differing physical and chemical properties of the individual bases of the biopolymer strand can in principle generate a measurable and reproducible modulation of the blockage current that allows an identification of the specific base sequence of the translocating biopolymer. These initially proposed systems suffer from a number of problems. For instance, some of the proposed systems require self-assembly of pore forming proteins on membranes (i.e. ahemolysin). Reproducibility of membranes an systems has been quite problematic. Secondly, commercial products require robustness not present in sensitive systems that require fluctuations of ionic currents for measurements. For these reasons, recent research has focused more on solid-state pore techniques that have an ability for high reproducibility and ease of fabrication.

Another method for detecting a biopolymer translocating through a nanopore has been proposed. This technique is based upon quantum mechanical tunneling currents through the portion of the translocating strand as it passes between a pair of electrodes. Measuring the magnitude of the tunneling current would be an electronic method of detecting the presence of a translocating biopolymer, and if the conditions were adequately controlled and the measurements sufficiently sensitive, the sequence of constituent bases could also be determined. One of the primary motivations for this approach is that typical tunneling currents in scanning tunneling microscopes are on the order of 1-10 nanoamps, which is two to three orders of magnitude larger than the ionic currents observed during polymer translocation of 2 nanometer nanopores, as described above (See Kasianowicz et al., “Characterization of individual polynucleotide molecules using a membrane channel”, Proc. Natl. Acad. Sci. USA, 93: 13770-13773, 1996).

The problem with such techniques is that they generally require nucleic acids that are free of other contaminants such as ribonucleic acid (RNA), proteins, or other molecules. Therefore, there is a need to purify or remove contaminants before the molecule to be sequenced reaches the nanopore. Otherwise, the extraneous material or contaminants will interfere with the quality of the overall results. Secondly, these high speed sequencing technologies require a way to easily and efficiently input the biomolecules to be sequenced. Of the present separation and sequencing devices to date none provide stability and ease of fabrication. These problems and others are addressed by the present invention.

### SUMMARY OF THE INVENTION

The invention provides an apparatus for identifying a chemical moiety from a sample solution. The system or apparatus comprises a substrate or housing having a channel with at least one microarray for capturing a chemical moiety from a sample solution, and a solid state nanopore system downstream from the substrate for identifying the chemical moiety received from the substrate channel after the chemical moiety has been released from the microarray.

The invention also provides a method for separating and identifying a chemical moiety. The method comprises contacting a solution comprising a target molecule to a probe positioned in a micro fluidic channel, binding the target molecule to the probe to separate the target molecule from the solution, releasing the target molecule off of the probe, and identifying or sequencing the target molecule released from the probe. Sequencing or identification may be by way of a solid state nanopore system. In particular the system may be ramped to perform resonant tunneling on the target of interest.

### BRIEF DESCRIPTION OF THE DRAWINGS

Embodiments of the invention will now be described with reference to the drawings, in which:

FIG. 1 shows a general block diagram of the present invention.

FIG. 2 illustrates a substrate carrying an array of the invention.

FIG. 3 is an enlarged view of a portion of FIG. 1 showing ideal spots or features.

FIG. 4 is an enlarged illustration of a portion of the substrate shown in FIG. 2.

FIG. 5 shows a perspective view of the present invention coupled to a nanopore system.

FIG. 6 shows a side elevation view of the present invention coupled to a nanopore system.

FIG. 7A shows a first embodiment of the nanopore system of the present invention.

FIG. 7B shows a side elevation of the first embodiment of the present invention.

FIG. 7C shows a second embodiment of the nanopore system of the present invention.

FIG. 7D shows a side elevation of the second embodiment of the nanopore system of the present invention.

FIG. 7E shows a third embodiment of the nanopore system of the present invention.

FIG. 7F shows a side elevation view of the third embodiment of the nanopore system of the present invention.

FIG. 7G shows a cross sectional view of a fourth embodiment of the nanopore system of the present invention.

FIG. 8 shows a perspective view of an embodiment of the present invention.

FIG. 9 shows a first step provided by the method of the present invention.

FIG. 10 shows a second step provided by the method of the present invention.

FIG. 11 shows a third step provided by the method of the present invention.

### DETAILED DESCRIPTION OF THE INVENTION

Before describing the invention in detail, it must be noted that, as used in this specification and the appended claims, the singular forms “a,” “an,” and “the” include plural referents unless the context clearly dictates otherwise. Thus, for example, reference to “an array” includes more than one “array”. Reference to an “electrode” or “substrate” includes more than one “electrode” or “substrate”. In describing and claiming the present invention, the following terminology will be used in accordance with the definitions set out below.

The term “adjacent” or “adjacent to” refers to a component or element that is near, next to or adjoining. For instance, an array may be adjacent to a nanopore system.

A “biopolymer” is a polymer of one or more types of repeating units. Biopolymers are typically found in biological systems (although they may be made synthetically) and particularly include peptides or polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A “peptide” is used to refer to an amino acid multimer of any length (for example, more than 10, 10 to 100, or more amino acid units). A biomonomer fluid or biopolymer fluid references a liquid containing either a biomonomer or biopolymer, respectively (typically in solution).

The term “in” refers to being “within” and/or a portion that may also be exterior to. For instance, a biopolymer “in” a nanopore may mean that the whole biopolymer is within the opening of the nanopore or only a small portion of the biopolymer is located near the nanopore with a substantial portion protruding exterior to the nanopore.

The term “nanopore” refers to any pore or hole between at least a pair of electrodes or a hole in a solid substrate. Nanopores can range in size from around 1 nm to around 300 nm. Most effective nanopores have been roughly 2-20 nms.

The term “portion” or “portion of a biopolymer” refers to a part, subunit, monomeric unit, portion of a monomeric unit, atom, portion of an atom, cluster of atoms, charge or charged unit.

The term “ramping potential” or “bias potential” refers to having the ability to establish a variety of different voltages over time. In certain cases this may be referred to as “scanning a voltage gradient” or altering a voltage gradient per unit of time. The ramping potential is produced by the “potential means”.

The term “resonant” or “resonant tunneling” refers to an effect where the relative energy levels between the current carriers in the electrodes are relatively similar to the energy levels of the proximal biopolymer segment. This provides for increased conductivity.

A “set” or “sub-set” of any item (for example, a set of features) may contain one or more than one of the item (for example, a set of clamp members may contain one or more such members). An “array”, unless a contrary intention appears, includes any one, two or three dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one that is to be evaluated by the other (thus, either one could be an unknown mixture of polynucleotides to be evaluated by binding with the other). An “array layout” refers collectively to one or more characteristics of the features, such as feature positioning, one or more feature dimensions, and some indication of a moiety at a given location. “Hybridizing” and “binding”, with respect to polynucleotides, are used interchangeably. When one item is indicated as being “remote” from another, this is referenced that the two items are at least in different buildings, and may be at least one mile, ten miles, or at least one hundred miles apart.

The term “substrate” or “substrate surface” are synonymous and refer to the material an electrode may be attached, comprise or be embedded in.

The term “symmetric” or “symmetrized” refers to similar tunneling barriers adjacent to both electrodes.

The term “translocation” or to “translocate” refers to movement from one side to another, or movement in a defined direction.

The term “tunneling” refers to the ability of an electron to move from a first position in space to a second position in space through a region that would be energetically excluded without quantum mechanical tunneling.

All patents and other cited references are incorporated into this application by reference.

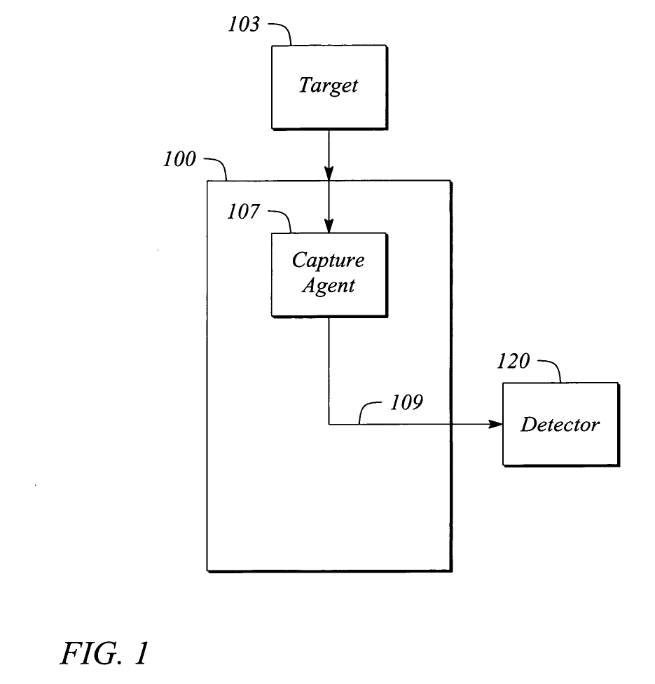

FIG. 1 shows a general block diagram of the present invention. The invention provides an apparatus for identifying a chemical moiety 100. The apparatus for identifying the chemical moiety 100 comprises a capture agent 107 for capturing a target 103, a transport device 109 for transporting the target 103 after it has been captured and released from the capture agent 107 and a detector such as a nanopore system 120 downstream from the transport device 109 for identifying, quantitating and sequencing the target 103. In certain embodiments the capture agent 107 may be positioned in, on, or adjacent to the transport device 109 or the nanopore system 120. In addition, the capture agent may comprise an array 112, the transport device 109 may comprise a first substrate 115 having a channel 118, and the nanopore system 120. The array 112 may be built directly into the channel 118 of the first substrate 115 or may be attached or inserted into position by mounting a slide or slides.

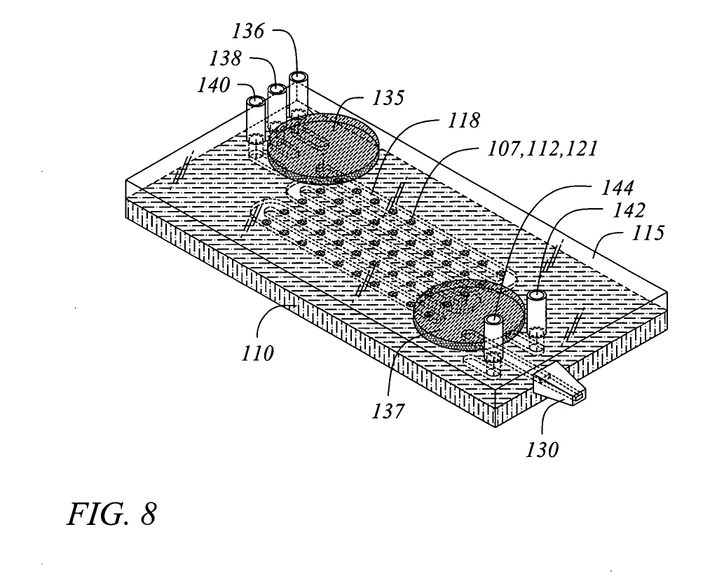

The first substrate 115 may comprise a number of different materials well know in the art. For instance, the first substrate 115 may comprise a material selected from the group consisting of metals, plastics, polycarbonate materials, rubber, silica or silicone based materials, and composite materials. The first substrate 115 may comprise flexible or non-flexible materials. As mentioned the first substrate 115 may comprise one or more channels 118. The channel 118 may comprise a micro fluidic channel 118 having one or more probes 121 (See FIG. 8). The channel 118 can be designed to be curved, linear or a variety of shapes and sizes. In addition, the probes 121 may comprise a variety of different biopolymers that may be oriented in a variety of different ways. The probes 121 may be positioned in random or non-linear arrangements. They also may be in linear arrangement, but this is not necessary. It is an important aspect of the invention that non-linear arrangements are possible with the present invention. Probe arrangement provides an efficient way to order targets 103 before capture and after release from the probes 121. Size and shape of the channel 118 is not important to the invention. The channels 118 need not be in a linear arrangement.

The sample may comprise one or more targets 103 that are transported through the channel 118. Transport may be accomplished through osmotic pressure, fluidic pressure, Brownian motion, diffusion, osmotic gradient, electro-osmotic gradient, gravity, capillary action, active or passive transport, electrophoresis, pressure, suction or creation of a vacuum or artificial vacuum or other physical or mechanical forces that are well know in the art. The technique is not important. However, functionally it is important that the technique efficiently regulates and allows the targets 103 to bind to the probes 121 that are attached or positioned in the channel 118 of the first substrate 115.

The array 112 may comprise a microarray or similar type device. As discussed, the array 112 may be constructed on the interior wall of channel 118 (See FIG. 8). In addition, the array 112 may be first designed on a slide and then inserted or mounted into the first substrate 115 that may appropriately position the probes 121 for hybridizing to target 103 in a sample. For simplicity the details of the array 112 are now described in relation to construction on a glass slide. The invention should not be limited to be interpreted to this embodiment and may also include a similar construction or design built on, in or attached to the first substrate 115.

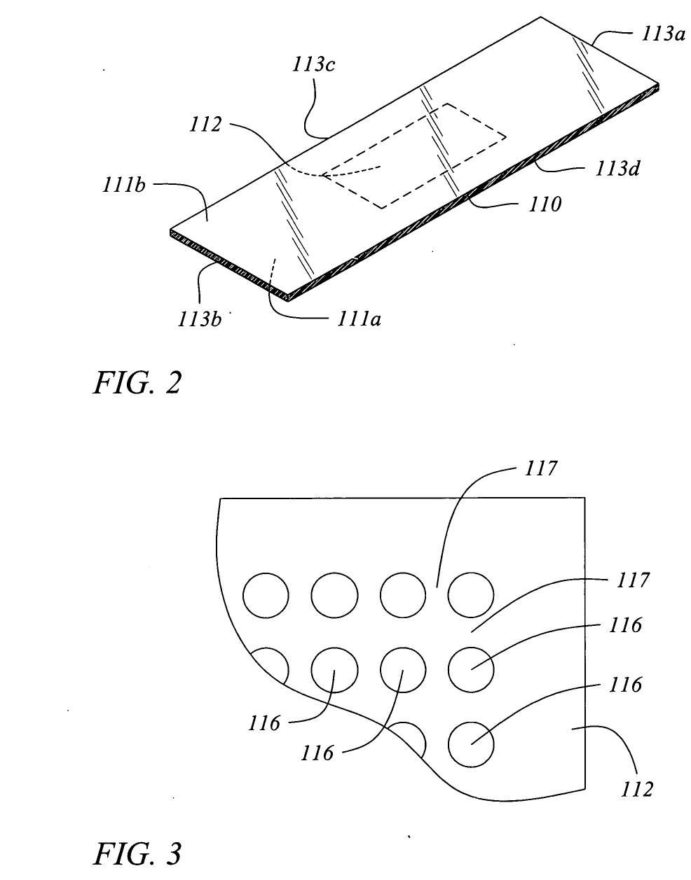

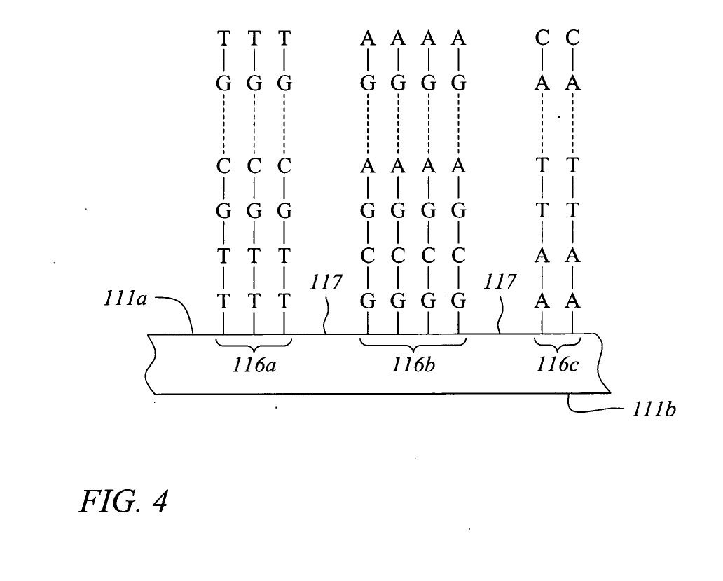

Referring now to FIGS. 2-4, typically the methods and apparatus of the present invention generate or use a contiguous planar second substrate 110 carrying an array 112 disposed on a rear surface 111a. It will be appreciated though, that more than one array (any of which are the same or different) may be present on the rear surface 111a, with or without spacing between such arrays. Note that one or more of the arrays 112 together will cover the substantial regions of the rear surface 111a, with regions of the rear surface 111a adjacent to the opposed sides 113c, 113d and the leading end 113a and the trailing end 113b of the second substrate 110. A front surface 111b of the second substrate 110 does not carry any of the arrays 112. Each of the arrays 112 can be designed for testing against any type of sample, whether a trial sample, reference sample, a combination of them, or a known mixture of polynucleotides (in which latter case the arrays may be composed of features carrying unknown sequences to be evaluated). The second substrate 110 may be of any shape, and any holder used with it adapted accordingly, although the second substrate 110 will typically be rectangular in practice. The array 112 contains multiple spots or features 116 of biopolymers in the form of polynucleotides. A typical array may contain from more than ten, more than one hundred, more than one thousand or ten thousand features, or even more than from one hundred thousand features. All of the features 116 may be different, or some or all could be the same. In the case where the array 112 is formed by the conventional in situ synthesis or deposition of previously obtained moieties, as described above, by depositing for each feature at least one droplet of reagent such as by using a pulse jet such as an inkjet type head, inter-feature areas 117 will typically be present which do not carry any polynucleotide. It will be appreciated though, that the inter-feature areas 117 could be of various sizes and configurations. Each feature carries a predetermined polynucleotide (which includes the possibility of mixtures of polynucleotides). As per usual, A, C, G, T represent the usual nucleotides. It will be understood that there may be a linker molecule (not shown) of any known types between the rear surface 111a and the first nucleotide. It should also be noted that bases or nucleotides may be modified or derivatized if desired.

The array 112 may comprise a biopolymer or in particular a nucleic acid or nucleotide sequence. Other biopolymers know in the art may be employed such as proteins, peptides, amino acids, nucleotides, nucleosides, nucleic acids, RNA, DNA, single stranded RNA, single stranded DNA, double stranded DNA or RNA etc., may be employed with the present invention. The target 103 or probe 121 sequence may be known or unknown. The biopolymers may be arranged in any of a number of orders and/or orientations on the array 112. This allows for the capture and release of biopolymers in a defined order or sequence.

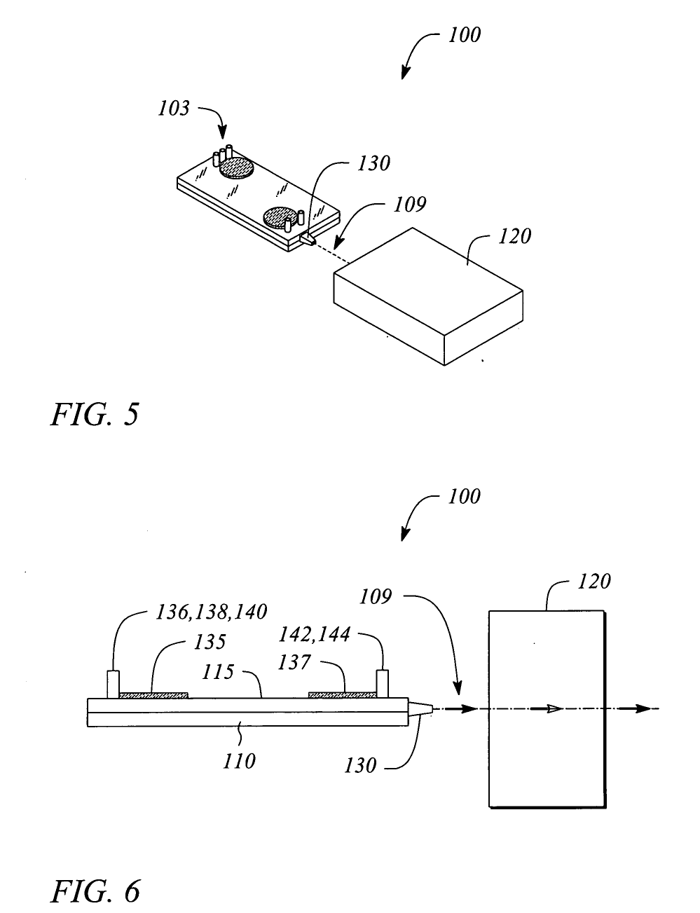

The nanopore system 120 is positioned downstream from the array 112 (See FIG. 5-6), note that the figures are for illustration purposes only and are not drawn to scale). Once the targets 103 have been separated from the sample they may then pass to the nanopore system 120. The nanopore system 120 is designed to receive the targets 103 and may record and quantify their nature, number, elution profile and order of elution from the array 112. Other parameters are also possible based on the nature of the nanopore system 120 that is employed. The nanopore system 120 may comprise a variety of devices and systems well known in the art. The nanopore system 120 may be coupled to the first substrate 115 either directly or through any number of conduits, channels, attachments or devices (not shown in FIGS.). These transport devices are important only to the extent that they allow for efficient sample transfer without loss of target 103.

Typical nanopore systems 120 used with the present invention may comprise and are not limited to devices disclosed and discussed in U.S. patent application Ser. No. 10/353,675 filed on Jan. 27, 2003 entitled “Apparatus and Method for Biopolymer Identification During Translocation Through a Nanopore” and Ser. No. 10/462,216 filed on Jun. 12, 2003 entitled “Nanopore with Resonant Tunneling Electrodes”. These disclosures are herein incorporated by reference in their entirety. Other solid-state nanopore devices known in the art may also be employed with the present invention.

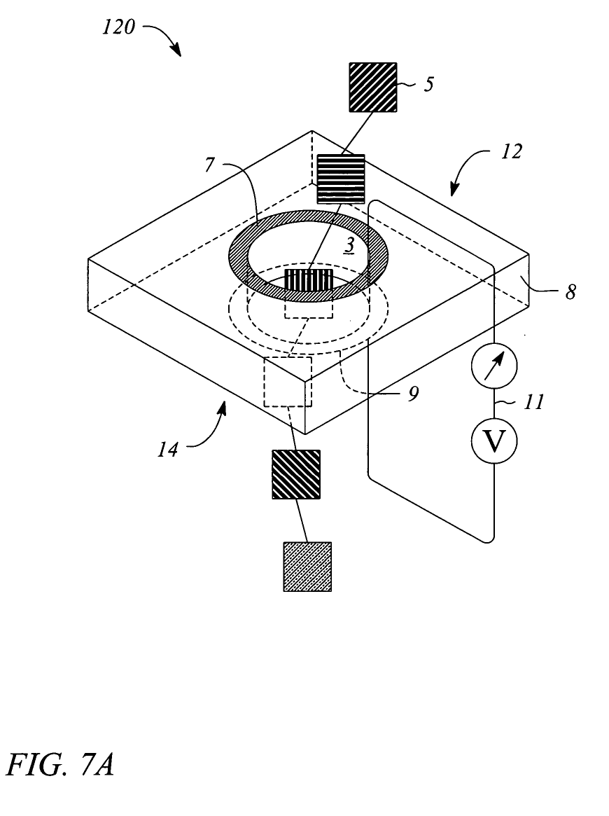

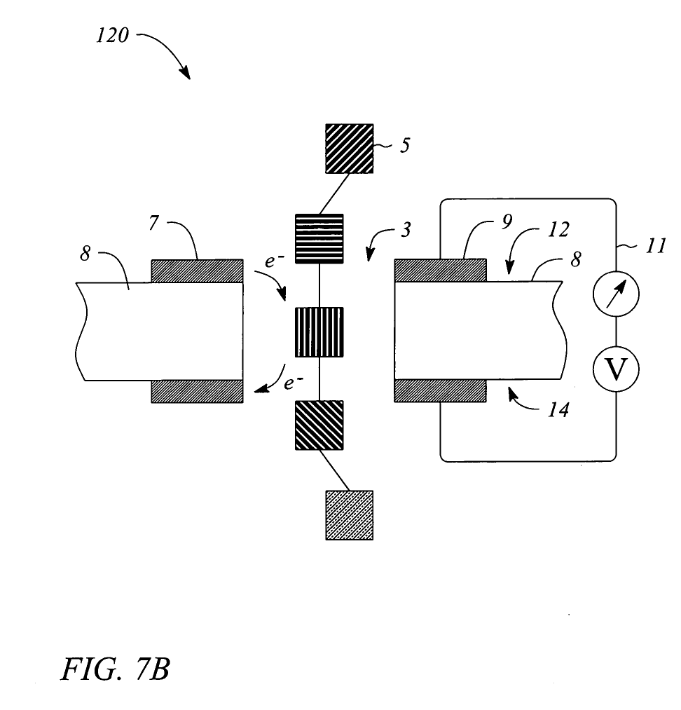

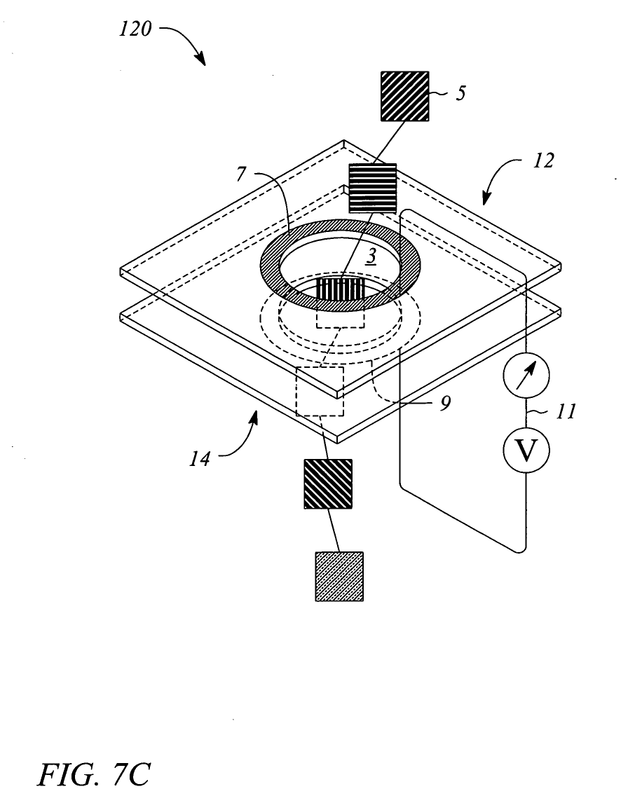

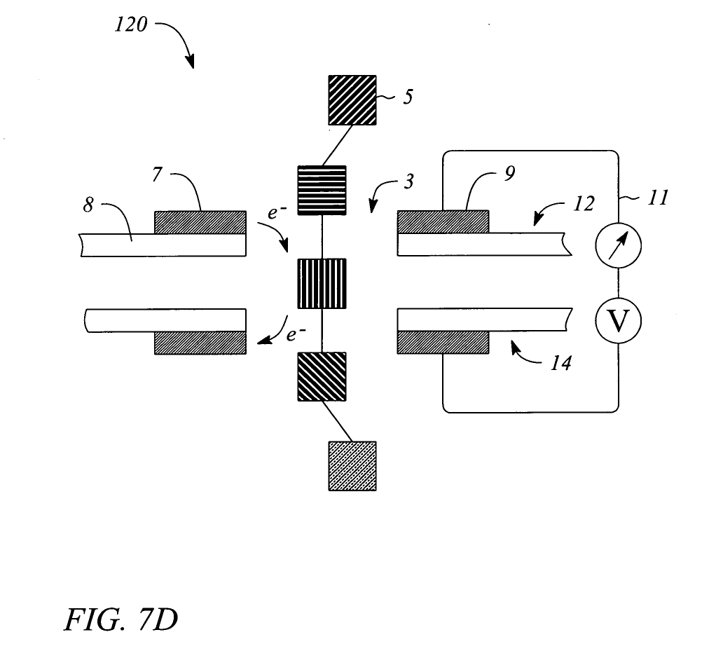

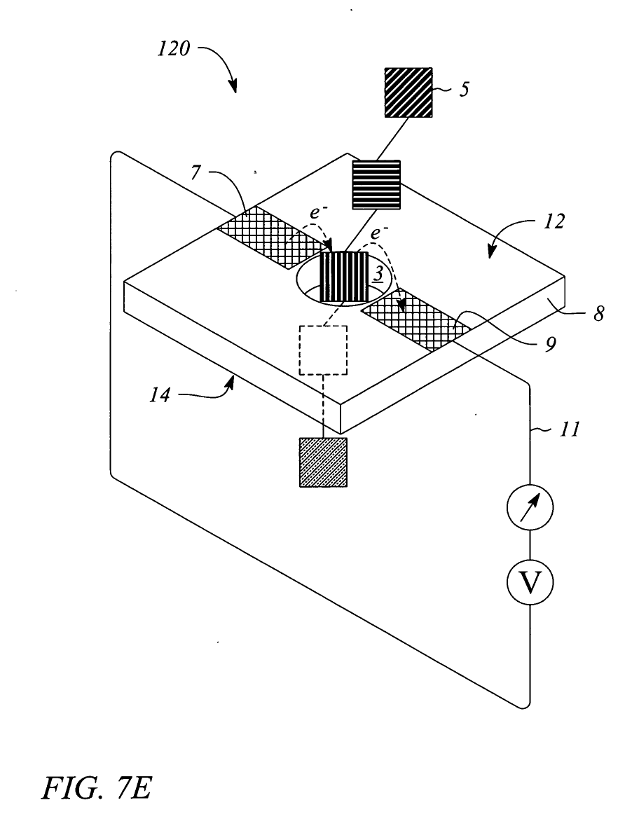

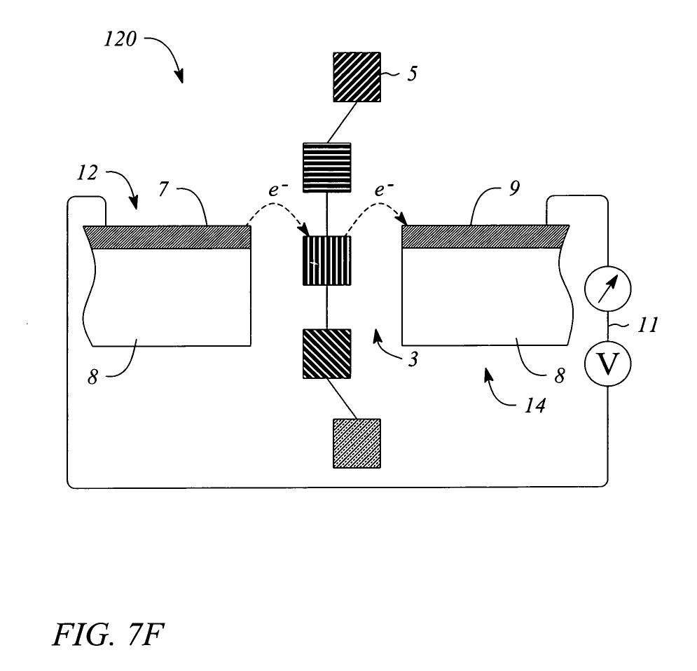

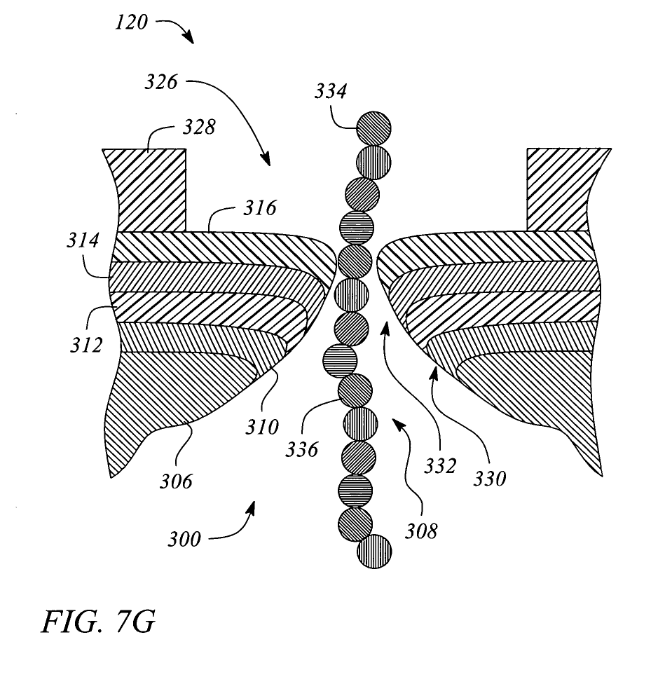

Referring now to FIGS. 7A-7G, the nanopore system 120 comprises a first electrode 7, a second electrode 9 and a voltage source 11. Either or both of the electrodes may be ring shaped. The first electrode 7 and the second electrode 9 are electrically connected to the voltage source 11. The second electrode 9 is adjacent to the first electrode 7 and spaced from the first electrode 7. A nanopore 3 may pass through the first electrode 7 and the second electrode 9. However, this is not a requirement of the invention. In the case that the optional substrate 8 is employed, the nanopore 3 may also pass through the substrate 8. Nanopore 3 is designed for receiving a biopolymer 5. The biopolymer 5 may or may not be translocating through the nanopore 3. When the optional substrate 8 is employed, the first electrode 7 and the second electrode 9 may be deposited on the substrate, or may comprise a portion of the substrate 8. In this embodiment of the invention, the nanopore 3 also passes through the optional substrate 8. Other embodiments of the invention may also be possible where the first electrode 7 and the second electrode 9 are positioned in the same plane (as opposed to one electrode being above or below the other) with or without the optional substrate 8 (See FIGS. 7E and 7F). The use of multiple electrodes and/or substrates are also within the scope of the invention.

The biopolymer 5 may comprise a variety of shapes, sizes and materials. The shape or size of the molecule is not important, but it must be capable of translocation through the nanopore 3. For instance, both single stranded and double stranded RNA and DNA may be used as a biopolymer 5. In addition, the biopolymer 5 may contain groups or functional groups that are charged. Furthermore, metals or materials may be added, doped or intercalated within the biopolymer 5 to provide a net dipole, a charge or allow for conductivity through the biomolecule. The material of the biopolymer must allow for electron tunneling between electrodes.

The first electrode 7 may comprise a variety of electrically conductive materials. Such materials include electrically conductive metals and alloys of tin, copper, zinc, iron, magnesium, cobalt, nickel, and vanadium. Other materials well known in the art that provide for electrical conduction may also be employed. When the first electrode 7 is deposited on or comprises a portion of the solid substrate 8, it may be positioned in any location relative to the second electrode 9. It must be positioned in such a manner that a potential can be established between the first electrode 7 and the second electrode 9. In addition, the bipolymer 5 must be positioned sufficiently close so that a portion of it may be identified or sequenced. In other words, the first electrode 7, the second electrode 9, and the nanopore 3 must be spaced and positioned in such a way that the bipolymer 5 may be identified or sequenced. This should not be interpreted to mean that the embodiment shown in FIG. 1 in any way will limit the spatial orientation and positioning of each of the components of the invention. The first electrode 7 may be designed in a variety of shapes and sizes. Other electrode shapes well known in the art may be employed. In addition, parts or curved parts of rings or other shapes may be used with the invention. The electrodes may also be designed in broken format or spaced from each other. However, the design must be capable of establishing a potential across the first electrode 7, and the nanopore 3 to the second electrode 9.

The second electrode 9 may comprise the same or similar materials as described above for the first electrode 7. As discussed above, its shape, size and positioning may be altered relative to the first electrode 7 and the nanopore 3.

The optional substrate 8 may comprise a variety of materials known in the art for designing substrates and nanopores. The substrate 8 may or may not be a solid material. For instance, the substrate 8 may comprise a mesh, wire, or other material that a nanopore may be constructed. Such materials may comprise silicon, silica, solid-state material such as Si₃N₄, carbon based materials, plastics, metals, or other materials known in the art for etching or fabricating semiconductor or electrically conducting materials. The solid substrate 8 may comprise various shapes and sizes. However, it must be large enough and of sufficient width to be capable of forming the nanopore 3 through it.

The nanopore 3 may be positioned anywhere on/through the optional substrate 8. As described above, the nanopore 3 may also be established by the spacing between the first electrode 7 and the second electrode 9 (in a planar or non planar arrangement). When the substrate 8 is employed, it should be positioned adjacent to the first electrode 7 and the second electrode 9. The nanopore may range in size from 1 nm to as large as 300 nms. In most cases, effective nanopores for identifying and sequencing biopolymers would be in the range of around 2-20 nm. These size nanopores are just large enough to allow for tranlocation of a biopolymer. The nanopore 3 may be established using any methods well known in the art. For instance, the nanopore 3, may be sculpted in the substrate 8, using argon ion beam sputtering, etching, photolithography, or other methods and techniques well known in the art.

The voltage source 11 may be positioned anywhere relative to the substrate 8, the nanopore 3, the first electrode 7 and the second electrode 9. The voltage source 11 should be capable of ramping to establish a voltage gradient between the first electrode 7 and the second electrode 9. A variety of voltage sources 11 may be employed with the present invention. A number of these voltage sources are known in the art. The voltage source 11 has the ability to ramp to establish a voltage gradient between the first electrode 7 and the second electrode 9. This is an important aspect of the present invention and for this reason is discussed in more detail below.

An optional means for signal detection may be employed to detect the signal produced from the bipolymer and voltage source 11. This means for signal detection may be any structure, component or apparatus that is well known in the art and that may be electrically connected to one or more components of the present invention.

Referring now to FIGS. 7C and 7D, a second embodiment of the invention, a series of separate substrates may be employed. For instance, a first substrate 12 and a second substrate 18 may be employed in place of the single substrate 8. In this embodiment of the invention, the first electrode 7 comprises first substrate 12 or a portion of this substrate. The electrode may be embedded, attached, layered, deposited, etched on the substrate or it may comprise all or a portion of the first substrate 12. Second electrode 9 comprises the second substrate 18 or a portion of the substrate. The electrode may be embedded, attached, layered, deposited, etched on the substrate or it may comprise all or a portion of the second substrate 18. The first substrate 12 is positioned adjacent to the second substrate 18. The figure shows the first substrate 12 positioned spatially above the second substrate 18. The first electrode 7 may comprise a first nanopore 3 while the second electrode 9 may comprise a second nanopore 3′. The first nanopore 3 of the first electrode 7 and the second nanopore 3′ of the second electrode 9 may have center points that are coaxially aligned to form a single contiguous pore that the biopolymer 5 may translocate through. It is within the scope of the invention that the nanopore 3 and the nanopore 3′ center points may be offset or spaced at relative angles and distances from each other. As described previously, the voltage source may be ramped to determine the portion of the biopolymer that is positioned within the electrode and nanopore.

Referring now to FIGS. 7E and 7F, a third embodiment of the present invention is provided. In this embodiment, the first electrode 7 and the second electrode 9 are positioned in the same plane. One or more optional substrates or electrodes may be employed. When the optional substrate 8 is not employed, the first electrode 7 and the second electrode 9 may be positioned adjacent to the nanopore 3. Although the figures show a pair of electrodes, the invention should not be interpreted to be limited to only this configuration. Various electrodes of varying shapes or sizes may be employed. Furthermore, it is anticipated that the invention comprises a number of similar or different electrodes capable of tunneling in a variety of directions and space (i.e. one, two and three dimensional space).

Referring now to FIG. 7G another embodiment of the present invention is shown. FIG. 7G illustrates a cross-sectional detail of an embodiment 300 of the present invention. Embodiment 300 is an embodiment in which the desired advantageous ring-shaped electrode structures of embodiment 200 are implemented. The fabrication process for embodiment 300 is illustrated and described in previous application Ser. No. 10/462,216 filed on Jun. 12, 2003 entitled “Nanopore with Resonant Tunneling Electrodes”. For more details on the construction of this apparatus please refer to the above mentioned application.

As seen in FIG. 7G, embodiment 300 comprises a nanopore 308 that is wide near its lower end and narrow near its upper end. Membrane 306 comprises a region of a material such as silicon dioxide, and the lower ring-shaped electrode 310 comprises a conductor such as platinum. Lower electrode 310 is formed in a manner that surrounds the perimeter of nanopore 308. On top of lower electrode 310a lower insulator layer 312 is placed in a manner that surrounds the perimeter of nanopore 308 and leaves exposed a perimeter portion 330 of electrode 310. On top of lower insulator layer 312, an upper electrode 314 is formed in a manner that surrounds the perimeter of nanopore 308. On top of upper electrode 314 an upper insulator 316 is placed in a manner that surrounds the perimeter of nanopore 308 and leaves exposed a perimeter portion 332 of electrode 314. Hole 326 in top insulator 328 provides access to the upper end of the nanopore for a biopolymer molecule 334 represented schematically as a string of beads.

One particular point along the biopolymer 334 represented as bead 336 is shown in a favorable position for resonant tunneling to occur. A voltage source, not shown, applies a time-varying potential difference between electrodes 310 and 314, via a circuit, not shown, similar to that used for embodiment 100, and the resulting time-varying current is measured by a current measuring means, not shown, similar to that shown in embodiment 100, in order to characterize that portion of biopolymer molecule 334 which happens to be in the favorable position for resonant tunneling to occur. For more information regarding the method of fabrication please refer to the reference Ser. No. 10/462,216 filed on Jun. 12, 2003 entitled “Nanopore with Resonant Tunneling Electrodes”.

FIG. 8 shows another view of the present invention. The apparatus for identifying a chemical moiety 100 is connected to the detector 120 by the transport device 109. The apparatus for identifying the chemical moiety 100 comprises the first substrate 115 having a channel 118 that comprises the capture agent 107. An input valve 135 and output valve 137 are coupled to the channel 118. Input valve 135 may be switched into various modes and inlet ports. For instance, there may be a sample inlet port 136, a wash buffer inlet port 138 and an elution buffer inlet port 140. Either or all of these inlet ports may be employed with the present invention. Other inlet ports may also be employed. The input valve 135 may be switched to allow flow from any one of these inlet ports to channel 118. At the opposite end of the channel 118 is the exit valve 137. Exit valve 137 may comprise or be connected to one or more wash outlet ports 142, salvage output ports 144, or electrospray tips 130. Exit valve 137 may be switched to the wash outlet port 142, salvage outlet port 144, or to electrospray tip 130.

While a significant benefit of the apparatus is to avoid the use of labels, it may be advantageous in certain instances to combine detection techniques. A labeled sample allows for fluorescent detection in-situ. A follow up elution or mass spectrometry measurement may provide more detailed information or confirmation of the measurements. Alternatively, it may be desirable to elute the target and analyze by gel eletrophoresis. This secondary and more expensive approach may be of interest for a reference laboratory or a central research facility.

Having described the apparatus of the invention, a description of the method of assembling or making the array hybridization apparatus is now in order.

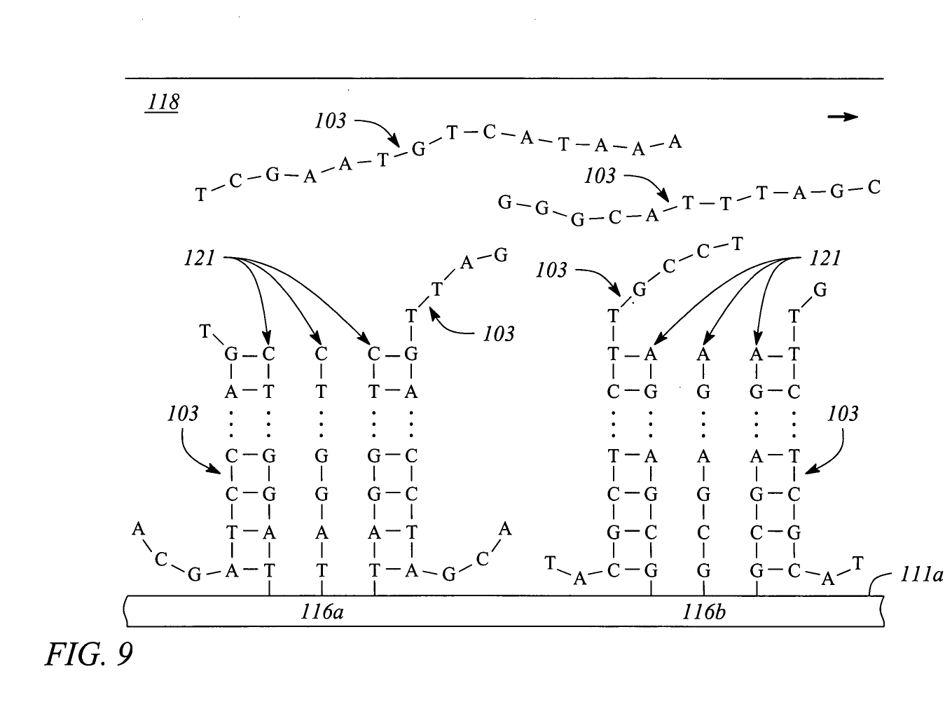

Referring now to FIGS. 9-11 the method of the present invention will now be discussed.

FIG. 9 shows the first step in the method of the present invention. A sample is introduced into the apparatus by any of a number of methods including injection, manual application etc. The figures show the sample being input by way of sample inlet port 136. There may be a sipper at the entrance of the channel which projects into the sample to draw the sample into the channel 118. A design of a sipper is described in the patent application entitled “Extensible Spiral for Flex Circuit”, Ser. No. 09/981,840, which is hereby incorporated by reference. Ideally, the entrance of the channel 118 may be coupled to a valve that allows for sample injection and then switches to inject a wash fluid. There is no requirement that the sample volume match the volume of the channel 118. It may be substantially larger or smaller. The sample may contain a target of interest. The sample with potential target of interest is moved past the probes of the array 112. The probes of the array 112 then capture the targets 103 and remove them from the sample. The apparatus can be designed to regulate the sample flow through the channel 118.

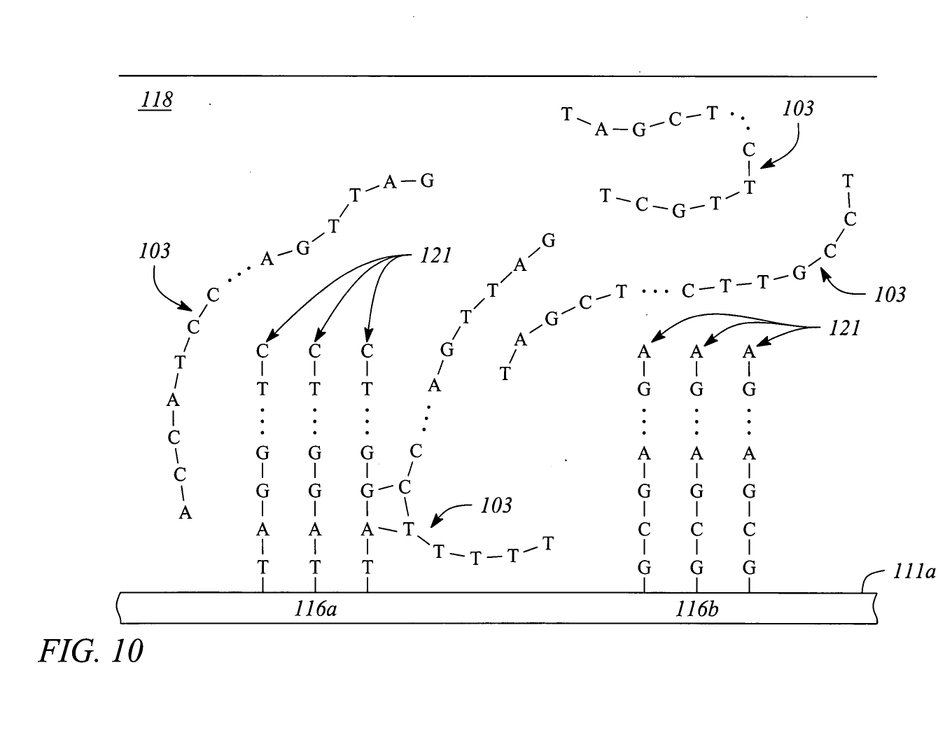

FIG. 10 shows the removal of the targets 103 from the sample as they bind to the array 112. The figure shows that the remainder of the sample or the bulk solution is then allowed to pass through the channel 118 of the substrate 110. The sample may be allowed to remain or cycle through channel 118 for minutes or hours as necessary to ensure adequate binding or hybridization to probe 121. The apparatus may include devices to control the time of sample exposure and heaters and coolers to control the temperature of the sample. After sufficient time has elapsed for binding or hybridization, the sample is washed out of the channel and wash buffers are introduced to remove any non-specifically bound target 103. The apparatus may control the timing and temperature of the wash buffers.

FIG. 10 shows the second step of the method of the present invention. In this step, an elution buffer or other agent may then be flushed through the apparatus. This allows the array 112 to release the targets 103 that have bound to the probes of the array 112. The targets 103 may also be released by raising the temperature of the solution or array around the probes 121. For instance, if the targets 103 and the probes 121 are nucleic acids, the temperature can be raised above their melting temperatures (Tm) to allow the nucleic acids to separate. This can be done for the entire channel driving off all of the captured targets 103 at once, or can be done serially in zone or by individual features to retain spatial segregation of the target eluants. The best methods will depend upon the diffusion characteristics of the targets 103 in a particular solution. Other mechanisms or methods for releasing the captured targets 103 may also be employed. For instance, the probes 121 may be held to the surface by a cleavable linker molecule. Thus, the entire probe/target duplex can be removed from the surface by cleaving the linker. Since the size of the channel 118 is small enough, the targets 103 maintain the same special ordering as they are bound to the probes 121. This process, therefore, serves as an effective separation technique. Depending on the specific construction of the apparatus, the captured targets 103 may be eluted from either end of the linear array. Therefore, the order of elution may be identical to the order of the probes 121 in the linear array or in the opposite order.

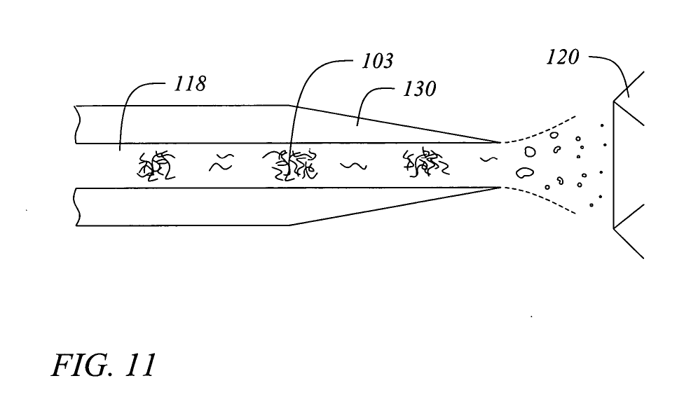

FIG. 11 shows the final step in the method of the present invention. In this step the targets 103 enter the detector or nanopore system 120 from the electrospray tip 130. They generally enter the detector or nanopore system 120 in the order in which they have eluted from the array 112. The detector may then record and determine the order, time, chemical composition, quantity or amount of target. By way of example, but not limitation, a micro-channel formed on a glass chip by photolithography and etching by methods known in the art. The cleaned interior of the channel is coated in poly-L-lysine. Pre-synthesized DNA oligomers are deposited in separate features along the interior of the channel and are bound by the poly-lysine. Oligos specific for controlled or known targets 103, called control features, are placed at the beginning and end of the array and at known locations along the linear array. There is a length of channel at either end which does not have features bound. After deposition, the poly-lysine is passivated by methods known in the art. The surface of the chip around the channel is coated with a thin layer of adhesive such as epoxy and a flat piece of glass comprising two holes is bonded to the chip to enclose the channel such that the holes are aligned with each end of the channel.

The RNA sample comprising unlabeled RNA and known control DNA targets is fragmented to lengths of approximately 200 mer using methods known in the art. Using a pipette, the channel is filled or nearly filled with the target. The chip is placed into an instrument that removably seals valve-controlled fluidic lines to each of the openings in the chip. In addition, the instrument controls the temperature of the chip.

The input valve 135 and exit valve 137 are adjusted so that each end of the chip is connected to a source of variable and controllable pressure that may be alternated to be above and below standard pressure as needed (See FIG. 6). The pressure sources are alternated to cause the sample to move back and forth in the channel such that the area with the features is never dry, but the sample is moved over the features. This sample movement process overcomes the limitations of diffusion and exposes more of the total sample to each feature. The mixing or sample movement process is continued continuously or periodically throughout the hybridization process. Additionally, the instrument heats the chip to the desired hybridization temperature, typically 37-65° C. and maintains the temperature for 1 to 24 hours, typically overnight.

After the hybridization period is complete, the instrument's valve at the inlet of the glass chip switches from the pressure pulses to the first wash fluid. The valve switches from pressure to the waste container. The first wash fluid is pumped through the channel driving the sample to the waste container and washing the array surface to remove unbound or non-specially bound sample. The wash fluid is generally not recirculated, although it may be. Next, the input valve 135 is switched to a second wash fluid that is added by way of wash buffer inlet port 138 as required by the assay. The wash fluid may be pumped through the channel for several minutes. During the wash protocol, the chip's temperature is generally returned to room temperature.

At the conclusion of the wash protocol, the input valve 135 is switched to the elution buffer. Elution buffer is pumped into the channel by way of the elution buffer inlet port 140 until the wash buffer is removed to waste (See FIG. 7). Then the exit valve 137 closes preventing any further fluid movement. The temperature of the system is raised above the melting point of the probes 121 driving the target from the probes 121 and into the elution buffer.

The exit valve 137 switches to the electrospray tip 130 and the electrospray mass spectrometer is then activated. The elution buffer is driven through the electrospray tip 130 into the mass spectrometer. The amount of target 103 eluted at each time point is quantitated. Since the flow rate is known, the signal at each time point can be correlated to each feature on the array for target identification. The control targets are used to establish the starting and ending points of the array as well as validate the timing along the array.

In summary, the method of the present invention operates for separating and detecting a chemical moiety such as a biopolymer. The steps of the method comprise contacting a sample comprising a target molecule to a probe positioned in a channel of a substrate, capturing the target molecule by contacting it with a probe, releasing the target molecule from the probe in a defined order and detecting the target molecule released from the probe in the defined order.

Clearly, minor changes may be made in the form and construction of the invention without departing from the scope of the invention defined by the appended claims. It is not, however, desired to confine the invention to the exact form herein shown and described, but it is desired to include all such as properly come within the scope claimed.

1. An apparatus for identifying a chemical moiety from a sample solution, comprising:
(a) a substrate having a channel with at least one array for capturing and releasing a chemical moiety from a sample solution; and
(b) a solid state nanopore system downstream from the substrate for identifying the chemical moiety received from the substrate channel after the chemical moiety has been released from the array, the nanopore system comprising
i. a first ring electrode,
ii. a second ring adjacent to the first ring electrode;
iii. a nanopore adjacent to the first ring electrode and the second ring electrode and positioned to allow the chemical moiety to be positioned in the first ring electrode and the second ring electrode; and
iv. a voltage source for electrically connecting the first ring electrode to the second ring electrode for applying a ramping potential from the first ring electrode, through a portion of the chemical moiety in the nanopore to a second ring electrode to produce a signal indicative of the portion of the chemical moiety.

2. An apparatus as recited in claim 1, further comprising a substrate for positioning the first electrode and the second electrode.

3. An apparatus as recited in claim 1, at least a first substrate for positioning the first electrode.

4. An apparatus as recited in claim 1, further comprising at least a second substrate for positioning the second substrate.

5. An apparatus as recited in claim 1, further comprising at least a first substrate for positioning a nanopore.

6. An apparatus as recited in claim 1, further comprising a means for signal detection for detecting the signal produced form the portion of the biopolymer.

7. An apparatus as recited in claim 1, where the channel is a micro fluidic channel.

8. An apparatus as recited in claim 1, wherein the array comprises a probe.

9. An apparatus as recited in claim 1, wherein the probe comprises a nucleic acid molecule.

10. An apparatus as recited in claim 1, wherein the probe comprises a protein molecule.

11. An apparatus as recited in claim 1, wherein the probe comprises a carbohydrate.

12. An apparatus as recited in claim 1, wherein the probe comprises a polysaccharide.

13. An apparatus as recited in claim 1, wherein the substrate comprises a material selected from the group consisting of silicon, plastic, rubber, glass, metal, and combinations thereof.

14. An apparatus as recited in claim 2, wherein the dimension of the micro fluidic channel is 100 microns or less.

15. An apparatus as recited in claim 1, wherein the target comprises an oligonucleotide.

16. An apparatus of claim 1, wherein the array comprises more than 100 features.

17. An apparatus of claim 1, wherein the substrate may be flexible or rigid.

18. An apparatus of claim 1, which further comprises valves in the channel that permit different fluids to be directed into the channel.

19. An apparatus of claim 1, which further comprises a temperature control device to provide a temperature controlled environment.

20. An apparatus of claim 1, which further comprises means to move the fluids through the array.

21. A method for separating and identifying a chemical moiety, comprising:
(a) contacting a solution comprising a target molecule to a probe positioned in a channel of a substrate;
(b) capturing the target molecule from the sample by contacting the target molecule to the probe;
(c) releasing the target molecule from the probe; and
(d) identifying the target molecule translocating through the nanopore system by applying a ramping electrical current form a first ring electrode through a portion of the chemical moiety to a second ring electrode to identify a portion of the chemical moiety positioned in the nanopore.

22. A method as recited in claim 24, wherein the electrical current is a tunneling current with an energy level that matches at least one conduction band energy of a portion of the chemical moiety.

23. A method as recited in claim 25, wherein the tunneling current is on resonance with the conduction band energies of a portion of the chemical moiety.

24. A method as recited in claim 24, further comprising translocating the chemical moiety through the nanopore to identify each of the translocating portions of the chemical moiety.

25. A method as recited in claim 24, wherein the order of release of the target molecule is the same as the order of binding of the target molecule to the probe.

26. A method as recited in claim 24, wherein the order of elution of the target molecule is opposite of the order of binding of the target molecule to the probe.

27. A method as recited in claim 24, wherein the step of releasing the target molecules involves heating portions of the array.

28. A method as recited in claim 24, wherein the target molecules are not labeled prior to introduction to the array.

29. A method as recited in claim 24, wherein the solution contacting the probes may comprise target molecules from more than one sample and the samples are differentially labeled.

30. An apparatus for identifying a chemical moiety from a sample solution, comprising:
(a) a substrate having a channel with at least one array for capturing a chemical moiety from a sample solution; and
(b) a solid state nanopore system downstream from the substrate for identifying the chemical moiety received from the substrate channel after the chemical moiety has been released from the array, the nanopore system comprising
i. a first electrode having a first nanopore,
ii. a second electrode adjacent to the first electrode having a second nanopore wherein the first nanopore of the first electrode is positioned with the second nanopore of the second electrode so that the chemical moiety may translocate through the first nanopore and the second nanopore; and
iii. a voltage source for electrically connecting the first electrode to the second electrode for applying a ramping potential from the first electrode, through a portion of the chemical moiety in the nanopore to a second electrode to produce a signal indicative of the portion of the chemical moiety.

31. An apparatus as recited in claim 33, wherein the first nanopore and the second nanopores have center points and wherein the center point of the first nanopore is positioned coaxially with the center point of the second electrode.

32. An apparatus as recited in claim 33, wherein the first electrode is positioned above the second electrode.

33. An apparatus as recited in claim 33, further comprising a substrate for positioning 30 the first electrode and the second electrode.

34. An apparatus as recited in claim 33, further comprising at least a second substrate for positioning the second electrode.

35. An apparatus for identifying a chemical moiety from a sample solution, comprising:
(a) a substrate having a channel with at least one array for capturing a chemical moiety from a sample solution; and
(b) a solid state nanopore system downstream from the substrate for identifying the chemical moiety received from the substrate channel after the chemical moiety has been released from the array, the nanopore system comprising
i. a first electrode,
ii. a second electrode spaced from the first electrode to define a nanopore between the first electrode and the second electrode, the nanopore designed for receiving a tranlocating chemical moiety, the first electrode being in electrical connection with the second electrode; and
iii. a voltage source for electrically connecting the first electrode to the second electrode for applying a ramping potential from the first electrode, through a portion of the chemical moiety in the nanopore to a second electrode to produce a signal indicative of the portion of the chemical moiety.

36. An apparatus as recited in claim 38, wherein the biopolymer is translocated in a stepwise fashion through the nanopore defined between the first electrode and the second electrode.

37. An apparatus for identifying a chemical moiety from a sample solution, comprising:
(a) a substrate having a channel with at least one array for capturing a chemical moiety from a sample solution; and
(b) a solid state nanopore system downstream from the substrate for identifying the chemical moiety received from the substrate channel after the chemical moiety has been released from the array, the nanopore system comprising
i. a first electrode layer having a first portion of the nanopore extending there through and exposing a first electrode edge;
ii. a first insulator layer adjacent to the first electrode layer, the first insulator layer having a second portion of the nanopore there through and defining a first insulator edge, the first insulator edge overhanging the first electrode edge;
iii. a second electrode layer adjacent to the first insulator layer, the second electrode layer having a third portion of the nanopore there through and defining a second electrode edge, the second electrode edge overhanging the first insulator edge; wherein the first electrode and the second electrode may be electrically ramped for sensing the chemical moiety.

38. A nanopore structure as recited in claim 40, further comprising a substrate adjacent to the first electrode.

39. A nanopore structure as recited in claim 40, wherein the first electrode edge defines a first diameter portion of the nanopore, and the first insulator edge defines a second diameter portion of the nanopore, the first diameter portion of the nanopore being smaller than the second diameter portion of the nanopore.

40. A nanopore structure as recited in claim 40, further comprising a second insulator layer contacting the second electrode layer and being adjacent to the nanopore, the second insulator layer having a fourth portion of the nanopore there through and defining a second insulator edge, the second insulator edge overhanging the second electrode edge.

41. A nanopore structure as recited in claim 43, wherein the second insulator edge defines a third diameter portion of the nanopore, the third diameter portion of the nanopore being smaller than the second diameter portion of the nanopore.

42. A nanopore structure as recited in claim 40, wherein the first electrode layer comprises a ring structure.

43. A nanopore structure as recited in claim 40, wherein the second electrode layer comprises a ring structure.

44. A nanopore structure as recited in claim 40, wherein both the first electrode layer and the second electrode layer comprise a ring structure.

45. A nanopore structure as recited in claim 40, further comprising an electric circuit.

46. A nanopore structure as recited in claim 48, wherein the electric circuit further comprises a voltage source for electrically connecting the first electrode layer with the second electrode layer and generating a potential between the first electrode layer and the second electrode layer for sensing a chemical moiety in the nanopore.

47. A nanopore structure as recited in claim 49, wherein the voltage source comprises a time varying voltage source.

48. A nanopore structure as recited in claim 50, where the electric circuit further comprises a current sensor for sensing a resulting current.

49. A nanopore structure as recited in claim 40, where the first electrode layer comprises a material selected from the group consisting of platinum, iridium, palladium, rhodium, gold, tin, copper, zinc, iron, magnesium, cobalt, nickel, vanadium and their alloys.

50. A nanopore structure as recited in claim 40, wherein the second electrode layer comprises a material selected from the group consisting of platinum, iridium, gold, tin, copper, zinc, iron, magnesium, cobalt, nickel, vanadium and their alloys.

51. A nanopore structure as recited in claim 40, wherein the nanopore is from 1 nanometer to 300 nanometers.

52. A nanopore structure a recited in claim 40, wherein the first insulator layer comprises a material selected from the group consisting of silicon dioxide, silicon nitride, oxynitride, platinum oxide, and aluminum oxide.

53. A nanopore structure a recited in claim 40, wherein the second insulator layer comprises a material selected from the group consisting of silicon dioxide, silicon nitride, oxynitride, platinum oxide, and aluminum oxide.
